(19) United States
(12) Patent Application Publication
(10) Pub. No.: US 2012/0064040 A1
(43) Pub. Date: Mar. 15, 2012

(54) SERUM FREE CULTURE MEDIUM AND SUPPLEMENT

(75) Inventors: Kara E. McCloskey, Merced, CA (US); Alicia A. Blancas, Merced, CA (US); Marcos E. Garcia-Ojeda, Merced, CA (US)

(73) Assignee: The Regents of the University of California

(21) Appl. No.: 13/152,185

(22) Filed: Jun. 2, 2011

Related U.S. Application Data
(60) Provisional application No. 61/351,249, filed on Jun. 3, 2010.

(51) Int. Cl. A61K 35/12; C12M 3/00; C12Q 1/02; C12N 5/0775; C12N 5/0789; C12N 5/10; C12N 5/071; C12N 5/0735

(52) U.S. Cl. 424/93.7; 435/405; 435/305.2; 435/325; 435/29; 435/289.1; 435/377

(57) ABSTRACT

Provided herein is a chemically defined method of endothelial cell (EC) derivation from embryonic stem cells (ESC). These progenitor cells are capable of low-density lipoprotein uptake, an important function of EC, and also express EC specific markers. By using chemically defined culture conditions, the reproducibility of the derivation as well as eliminate the possibility of unknown contaminants such as undefined growth factors and sundry animal proteins is improved. The differentiated cells can then be applied to a myriad of potential therapies such as tissue engineered vascular grafts, cardiac patches, and pre-vascularized tissue transplants.

### CROSS-REFERENCE TO RELATED APPLICATIONS

This application claims the benefit under 35 U.S.C. §119(e) of U.S. Provisional Application Ser. Nos. 61/351,249, filed Jun. 3, 2010 the contents of which is incorporated by reference in its entirety into the present disclosure.

### STATEMENT OF FEDERAL SUPPORT

This invention was made with Government support under grant number F31 HL087716 awarded by the National Institutes of Health. The Government has certain rights in the invention.

### FIELD OF THE INVENTION

The present invention relates generally to the field of derivation of endothelial cells from embryonic stem cells using chemically defined medium.

### BACKGROUND

Throughout this disclosure, various technical and patent publications are referenced to more fully describe the state of the art to which this invention pertains. The publications may be referenced by an Arabic numeral. The full bibliographic citation for these publications are found in the section of this document immediately preceding the claims. All publications are incorporated by reference, in their entirety, into this application.

Cardiovascular disease is the leading cause of death in the United States. Many cell-based therapies have been explored, particularly those involving the use of stem cells. The pluripotent nature of embryonic stem cells (ESC) facilitates their differentiation into all of the mature cellular lineages in the body.

Embryonic stem cells are isolated from the inner cell mass (ICM) of an embryonic blastocyst. They are pluripotent, retain the capacity to self-renew, as well as differentiate into cells from all three germ layers. Although it is possible to obtain stem cells from adult sources such as bone marrow and adipose tissue, adult cells exhibit limited pluripotency compared with embryonic stem cells. Adult stem cells also can be difficult to identify, isolate and expand in culture. Murine ESC are an especially attractive cell culture system because they can be maintained undifferentiated in culture easily.

Endothelial cells are highly dynamic cells involved in regulating a variety of vascular functions. Endothelial cells regulate blood pressure through controlling vasodilation and vascoontriction via synthesis of nitric oxide. Endothelial cells also regulate the permeability of the endothelium, and activate and recruit leukocytes in response to inflammation. It is well known that endothelial cells help inhibit platelet adhesion and clotting. Endothelial cells are also the key cells involved in new blood vessel growth and assembly.

Vascular endothelial cells or endothelial progenitor cells derived from stem cells could potentially lead to a variety of clinically relevant applications [1]. These cells could be used in therapeutic strategies for the repair and revascularization of ischemic tissue in patients exhibiting vascular defects [2]. Endothelial progenitor cell transplantation has been shown to induce new vessel formation in ischemic myocardium and hind limb [2-4]. Since endothelial cells inhibit platelet adhesion and clotting, endothelial cells lining the lumen of a synthetic or tissue-engineered vascular graft or re-endothelization of injured vessels can aid in patency of vascular grafts [2,5]. Moreover, because endothelial cells line the lumen of blood vessels and can release proteins directly into the blood stream, they are ideal candidates to be used as vehicles of gene therapy. Lastly, endothelial cells are a key player in the development of vascularizing tissue-engineered materials [6]

Methods of successful differentiation of EC from ESC and adult stem cells in vitro have been previously described [7-10]. One common method used in the derivation of EC from ESC involves the formation of a three-dimensional (3-D) mass of tissue called an embryoid body. This structure can contain cell types from all germ layers, but controlling the cell-cell contacts and cell microenvironment is difficult. However, a two-dimensional (2-D) monolayer system allows for easier cell visualization additionally enabling morphological examinations and better control over the cells' microenvironment [11]. Endothelial promoting growth factors, such as vascular endothelial growth factor (VEGF), can be added to the differentiation medium, but also are present in unknown amounts in serum.

Applicants and others have published methods for the differentiation of EC from ESC using fetal bovine serum [12-14]. The differentiated cells are purified by fluorescence activated cell sorting (FACS), magnetic cell sorting (MACS), or by manually picking cells from culture plates. When inducing ESC to differentiate towards endothelial cells, current methods involve LIF removal in cultures with serum plus growth factors specific for the desired cell type [25, 31, 34]. However, serum is not a chemically defined reagent and can vary from batch-to-batch. Differentiation methods using serum lend an element of unpredictability in culture conditions and introduce factors that cannot be well-controlled. Most researchers attempt to secure an entire batch of serum from a manufacturer, ensuring the research is at least reproducible within one's own laboratory. Unfortunately, once the serum supply is exhausted, it becomes necessary to perform labor intensive batch testing by running replicates of experiments in order to determine which batch yields the most favorable results. Optimal conditions for stem cell differentiation would also be difficult to precisely reproduce in other laboratories using different batches of serum.

For these reasons, serum replacements have been successfully incorporated in the methods of ESC maintenance and some differentiation methods [15-17]. Although most formulations of serum replacements are proprietary, they are generally free from animal components and demonstrate negligible batch-to-batch variation, thereby facilitating experimentation in a chemically defined condition. By using a chemically defined medium, it is possible to more accurately control the cell's microenvironment and better evaluate the response of particular biochemical or physical signals. In addition, comparisons between primary aortic endothelial cells and ESC-derived EC have shown that stem cell derived-EC express a wide range of EC markers, but may lack some of the functions observed in normal aortic EC such as decreased uptake of low density lipoprotein (LDL) [18]. The maturation and function of tissue-specific cells might also be improved by using chemically defined medium formulations for directed EC differentiation.

Thus, a need exists to provide compositions and methods to facilitate the differentiation of cells into defined cell lineages which in turn, may be used therapeutically and diagnostically. This invention satisfies this need and provides related advantages as well.

### SUMMARY

Applicants have developed a chemically defined method of endothelial cell (EC) derivation from embryonic stem cells (ESC). These progenitor cells are capable of low-density lipoprotein uptake, an important function of EC, and also express EC specific markers. By using chemically defined culture conditions, Applicants can improve the reproducibility of the derivation as well as eliminate the possibility of unknown contaminants such as undefined growth factors and sundry animal proteins. The differentiated cells can then be applied to a myriad of potential therapies such as tissue engineered vascular grafts, cardiac patches, and pre-vascularized tissue transplants.

Thus, in one aspect, this invention provides a cell culture medium comprising, or alternatively consisting essentially of, or yet further consisting of, from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml of a basic fibroblast growth factor (bFGF) or an equivalent thereof; from about 0.5 mM to about 5 mM of L-glutamine; from about 0.5× to about 3× of non essential amino acids; from about 50 to about 300 units/ml of penicillin; from about 50 to about 300 units/ml of streptomycin; and from about 0.01 mM to about 2 mM of 2-mercaptoethanol, admixed in a basal medium comprising, or alternatively consisting essentially of, or yet further consisting of, from about 50% to about 80% (w/w) of Alpha MEM and from about 20% to about 50% of DMEM.

In another aspect, this invention provides a cell culture medium comprising, or alternatively consisting essentially of, or yet further consisting of, from about 0.5× to about 3× of a chemically defined serum replacement; from about 10 ng/ml to about 170 ng/ml of a vascular endothelial growth factor (VEGF) or an equivalent thereof; from about 10 ng/ml to about 100 ng/ml basic of a fibroblast growth factor (bFGF) or an equivalent thereof; from about 0.5 mM to about 5 mM of a L-glutamine; from about 0.5× to about 3× of non essential amino acids; from about 50 units/ml to about 300 units/ml penicillin; from about 50 units/ml to about 300 units/ml of streptomycin; and from about 0.01 mM to about 2 mM of 2-mercaptoethanol, admixed in a basal medium comprising from about 50% to about 80% (w/w) of Alpha MEM and from about 20% to about 50% of DMEM.

Yet further provided is a cell culture medium comprising, or alternatively consisting essentially of, or yet further consisting of, from about 10% to about 30% (w/w) of a chemically defined serum replacement; from about 50 units/ml to about 300 units/ml of penicillin; from about 50 units/ml to about 300 units/ml streptomycin; from about 0.5 to about 3× of non essential amino acids; from about 0.5 mM to about 5 mM L-glutamine; from about 0.01 mM to about 2 mM 2-mercaptoethanol; from about 10 ng/ml to about 100 ng/ml of a vascular endothelial growth factor (VEGF) or an equivalent thereof; and from about 1 ng/ml to about 20 ng/ml bone of a morphogenetic protein 4 (BMP4) or an equivalent thereof or about 3 ng/ml to about 200 ng/ml of an Activin A [19-21] or an equivalent thereof, admixed in Alpha MEM.

The compositions of this invention are useful to differentiate an isolated embryonic stem cell, an embroyid body, a parthenogenetic stem cell or an induced pluripotent stem (iPS) cell to an endothelial cell progenitor or an endothelial cell or a population of endothelial progenitors or endothelial cells. In one aspect, the population of cells provided by use of the compositions and methods of this invention are substantially homogeneous. The isolated stem cell is of animal origin, e.g., a mammalian cell, e.g., a human cell, a simian cell, a bovine cell or a murine cell. The cells can be cultured cells, e.g., available from the American Tissue Culture Collection (ATCC, Bethesda Md. or Wicell, Madison. Wis., for example) or isolated from an animal or human subject using methods known to those skilled in the art.

Also provided is a composition that comprises, or alternatively consists essentially of, or yet further consists of, an additional carrier, e.g., a solid phase carrier such as a stem cell plate or a biocompatible scaffold which may or may not be coated with a material or composition such as one that facilitates cell/plate interations.

This invention also provides a method of preparing the composition by admixing the components as identified herein.

The compositions of the invention are useful for differentiating, in vitro, an isolated embryonic stem cell, an IPSC, adult bone marrow stem cell, a cord blood stem cell, an parthenogenetic stem cell or an isolated embryoid body into endothelial progenitor cells or an endothelial population of same, by contacting an effective amount of a composition as described above with the cell to be differentiated. In one aspect, the medium components are pre-mixed to the effective amount and then added to the cell(s) or base medium or alternatively, the components are added to the cells in base medium in amount which brings the concentration of the components to the effective amount(s) described herein. These compositions or components are contacted with the cell(s) or population(s) and then cultured under suitable conditions and for an effective amount of time to differentiate the cells. For the purpose of illustration only, the effective amount of time includes, for example, for more than one day, or alternatively for two days, or for three days or more, prior to media change. In one aspect, the media can be changed and the cells continued to be cultured. Methods of determining when the cells have been differentiated are known in the art and include for example, the expression of endothelial specific cell markers or by observing physiological characteristics. The cell populations prepared by the methods and compositions of this invention are substantially homogeneous.

### BRIEF DESCRIPTION OF THE FIGURES

FIG. 1 illustrates Flk-1 expression at several initial induction times (2-5 days) on various substrates. Error bars represent SEM.

FIG. 2 illustrates viable and adherent cells from initial (2 day) induction to Flk-1+ vascular progenitor cells. Dashed line represents initial number of cells seeded (50,000). Error bars represent SEM.

FIG. 3 depicts a manual selection apparatus. Cobblestone colonies are aspirated, replated, and expanded for analysis.

FIG. 4 shows flow cytometry analysis of EC markers expressed by the derived EC lines.

FIG. 5 illustrates LDL uptake assay for (A) dEC3-11 and (B) dEC3-13.

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2″ edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

The term “isolated” means separated from constituents, cellular and otherwise, in which the cell, tissue, polynucleotide, peptide, polypeptide, protein, antibody or fragment(s) thereof, which are normally associated in nature. For example, an isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart. An isolated cell is a cell that is separated form tissue or cells of dissimilar phenotype or genotype.

A “chemically defined serum replacement” intends, a “biochemically defined serum replacement”, or simply a “defined serum replacement” intends a serum replacement, composition of which is known or can be substantially ascertained. Like serum, a chemically defined serum replacement can be used to support cell culture, but unlike serum, the composition of a chemically defined serum replacement is consistent across lots. In some embodiments, a chemically defined serum replacement is a synthetic serum replacement. Examples of chemically defined serum replacement include, but are not limited to, Nutridoma CS (Roche Applied Science, Indianapolis, Ind.), Nutridoma SP (Roche Applied Science, Indianapolis, Ind.), TCH™ (MP Biomedicals, LLC, Solon, Ohio), KnockOut™ Serum Replacement (Invitrogen Corporation, Carlsbad, Calif.), or CDM-HD Serum Replacement (FiberCell Systems Inc. Frederick, Md.).

As used herein, “stem cell” defines a cell with the ability to divide for indefinite periods in culture and give rise to specialized cells. At this time and for convenience, stem cells are categorized as somatic (adult), embryonic, induced pluripotent stem cells and/or parthenogenetic stem cells (see Cibelli et al. (2002) Science 295(5556):819; U.S. Patent Publ. Nos. 20100069251 and 20080299091). A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also know as WA01) cell line available from WiCell, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An-induced pluripotent stem cell (iPSC) is an artificially derived stem cell from a non-pluripotent cell, typically an adult somatic cell, produced by inducing expression of stem cell specific genes. A parthenogenetic stem cell is one which was generated in vitro by parthenogenetic development cells from an egg, without the use of male sperm.

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue.

The terms “culturing” or “incubating” refer to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell.

As used herein, the term “differentiates or differentiated” defines a cell that takes on a more committed (“differentiated”) position within the lineage of a cell. “Dedifferentiated” defines a cell that reverts to a less committed position within the lineage of a cell.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “composition” is also intended to encompass a combination of a compound or composition and another carrier, e.g., a solid support such as a culture plate or biocompatible scaffold, inert (for example, a culture plate) or active, such as an adjuvant, diluent, binder, stabilizer, buffers, salts, lipophilic solvents, preservative, adjuvant or the like. Carriers also include pharmaceutical excipients and additives such as proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker and or by observing physiological characteristics of the cell or cells.

The term “effective amount” refers to a concentration or amount of a reagent or composition, such as a composition as described herein, cell population or other agent, that is effective for producing an intended result, including cell growth and/or differentiation in vitro or in vivo, or for the treatment of a disease or condition as described herein. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or composition to achieve its intended result, e.g., the differentiation of cells to a pre-determined cell type.

The term patient or subject refers to animals, including mammals, preferably humans, who are treated with the pharmaceutical compositions or in accordance with the methods described herein. Other animals include, simians, bovines, ovines, equines, canines, felines, and murines.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

As used herein, a “pluripotent cell” defines a less differentiated cell that can give rise to at least two distinct (genotypically and/or phenotypically) further differentiated progeny cells. In another aspect, a “pluripotent cell” includes a Induced Pluripotent Stem Cell (iPSC) which is an artificially derived stem cell from a non-pluripotent cell, typically an adult somatic cell, produced by inducing expression of stem cell specific genes. Such stem cell specific genes include, but are not limited to, the family of octamer transcription factors, i.e. Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al. (2007) Nature 448:260-262; Yu et al. (2007) Science advance online publication 20 Nov. 2007; and Nakagawa et al. (2007) Nat. Biotechnol. Advance online publication 30 Nov. 2007.

An “endothelial cell” is a highly dynamic cell involved in regulating a variety of vascular functions. Endothelial cells regulate blood pressure through controlling vasodilation and vascoontriction via synthesis of nitric oxide. Endothelial cells also regulate the permeability of the endothelium, and activate and recruit leukocytes in response to inflammation. It is well known that endothelial cells help inhibit platelet adhesion and clotting. Endothelial cells are also the key cells involved in new blood vessel growth and assembly. Markers for identification of a terminally differentiated endothelial cell include, but are not limited to: 7B4 antigen, ACE (angiotensin-converting enzyme), Ang1, Ang2, Ang3, Ang4, ANGPTL2, ANGPTL3, ANGPTL4, ANGPTL5, ANGPTL6, ANGPTL7, BNH9/BNF13, CD31 (PECAM-1), CD34, CD54 (ICAM-1), CD62P (p-Selectin GMP140), CD105 (Endoglin), CD146 (P1H12), sCD146, D2-40, E-selectin, EN4, Endocan, Endoglin (CD105), Endoglyx-1, Endomuci, Endosialin (tumor endothelial marker 1, TEM-1, FB5), Eotaxin-3, EPAS1 (EndothelialPAS domain protein 1), Factor VIII related antigen, FB21, Flk-1 (VEGFR-2, KDR), Flt-1 (VEGFR-1), GBP-1 (guanylate-binding protein-1), GRO-alpha, Hex, ICAM-2 (intercellular adhesion molecule 2), LYVE-1, MECA-32, MECA-79 MRB (magic roundabout), Nucleolin, PAL-E (pathologische anatomie Leiden-endothelium), RPTPmu (Receptor protein tyrosine phosphatase mu), RTKs sVCAM-1, TEM1 (Tumor endothelial marker 1), TEM5 (Tumor endothelial marker 5), TEM7 (Tumor endothelial marker 7), TEM8 (Tumor endothelial marker 8), Thrombomodulin (TM, CD141), TIE-1, TIE-2, VCAM-1 (vascular cell adhesion molecule-1) (CD106), VE-cadherin (CD144), VEGF (Vascular endothelial growth factor), and vWF (von Willebrand factor). Also to be included are the soluble forms of these markers. Not all EC will express all markers at all times. Expression can vary.

An endothelial progenitor cell is a cells or populations of cells that are committed to the endothelial lineage, but are not terminally differentiated. In addition, lower expression levels of the aforementioned markers is typical.

An arterial endothelial cell is a specialized endothelial cell population that lines the lumen of arteries. Both arterial and arterial progenitors will express varying levels of the aforementioned markers and will also express of the following arterial EC markers: Alk1, Bmx (Rajantie et al. 2001), CD44, Connexin37, Connexin40, CXCR4, Delta-like4, Depp, ephrinB2, GFBP-5P, Jagged1, Neuropilin1, Notch1, Notch4, and Unc5b [22].

Venous endothelial cells are specialized endothelial cells that line the lumen of veins. Venous EC and EPC also express various levels of EC markers disclosed above in addition to the expression indicated above, venous expression is of the following venous EC markers: COUP-TFII, EphB4, and Neuropilin2 [22].

A vascular endothelial cell is a specialized type of epithelial cell that lines the lumen of the vessels, capillaries, arteries, and veins throughout an organism. Markers for identification of a vascular endothelial progenitor are provided above. The cells can also express lymphatic cellular markers during development and differentiation. A non-limiting list of lymphatic specific EC markers include: CCL21, LYVE1, Neuropilin2, Podoplanin, Prox1, and VEGFR3. [22].

A mesodermal progenitor cell is a cell that is committed to the mesodermal lineage, as opposed to ectoderm or endoderm. Mesodermal tissues include muscle, cardiovascular tissue, and hematopoietic cells. Markers for identification of a mesodermal progenitors include but are not limited to: Flk-1, Brachyury, CD31 (PECAM1), CD325 (M-cadherin), CD34, (Mucosialin), NF-YA and Sca-1 (Ly6A/E).

“Vascular endothelial growth factor (VEGF)” refers to a family of growth factors that are important signaling proteins involved in both vasculogenesis and angiogenesis. The protein is commercially available from a number of vendors (e.g., ProSpecBio.com) or it can be recombinantly or chemically produced using the amino acid or polynucleotide information that is publicly available, e.g., GenBank: AAA35789.1 (accessed on Apr. 6, 2010). An “equivalent thereof” intends an equivalent or modified protein, such as a fragment, that has the same or similar activity to the naturally occurring protein.

“Basic fibroblast growth factor (bFGF)” is a protein that has been shown to be important in the regeneration of granulation tissue and to maintain embryonic stem cells in an undifferentiated state. The protein is commercially available from a number of vendors or it can be recombinantly or chemically produced using the amino acid or polynucleotide information that is publicly available, e.g., GenBank NP_{—}001997 (accessed on Apr. 6, 2010) or NP_{—}032032 (accessed on Apr. 6, 2010). An “equivalent thereof” intends an equivalent or modified protein, such as a fragment, that has the same or similar activity to the naturally occurring protein.

“Basal medium” is an unsupplemented medium which promotes the growth of many types of microorganisms and cells in culture. Non-limiting examples of basal medium include Basal Medium Eagle, Dulbecco's Modified Eagle Medium and F-10 Nutrient Mixture. They are commercially available, e.g., Biological Industries, Invitrogen.

“Bone morphogenetic protein 4 (BMP4)” is a protein that belongs to the TGF-beta superfamily of proteins. It is involved in bone and cartilage development. In human embryonic development, it is a factor in the early differentiation of the embryo and establishment of a dorsal-ventral axis. The protein is commercially available from a number of vendors or it can be recombinantly or chemically produced using the amino acid or polynucleotide information that is publicly available, e.g., GenBank NM_{—}001202 (human) NM_{—}007554 (mouse) (accessed on Apr. 6, 2010) or NP_{—}001193 (human) or NP_{—}031580 (mouse) (accessed on Apr. 6, 2010). An “equivalent thereof” intends an equivalent or modified protein, such as a fragment, that has the same or similar activity to the naturally occurring protein.

Activin A is multifunctional an known to enhance FSH biosynthesis and secretion, mesoderm induction and neural cell differentiation. The protein is commercially available from a number of vendors (e.g., R&D Systems) or it can be recombinantly or chemically produced using the amino acid or polynucleotide information that is publicly available, e.g., in the GenBank database. An “equivalent thereof” intends an equivalent or modified protein, such as a fragment, that has the same or similar activity to the naturally occurring protein.

Alpha MEM and DMEM intend basal mediums commercially available from a number of commercial vendors, e.g., Invitrogen.

### Compositions

In one aspect, this invention provides a culture medium comprising, or alternatively consisting essentially of, or yet further consisting of, from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml basic fibroblast growth factor (bFGF) or an equivalent thereof; from about 0.5 mM to about 5 mM of L-glutamine; from about 0.5× to about 3× of non essential amino acids; from about 50 units/ml to about 300 units/ml of penicillin; from about 50 units/ml to about 300 units/ml of streptomycin; and from about 0.01 mM to about 2 mM of 2-mercaptoethanol, admixed in a basal medium comprising from about 50% to about 80% (w/w) of Alpha MEM and from about 20% (w/w) to about 50% (w/w) of DMEM.

In an alternative embodiment, the invention provides a culture medium comprising, or alternatively consisting essentially of, or yet further consisting of, from about 0.5× to about 3× of a chemically defined serum replacement; from about 10 ng/ml to about 170 ng/ml of a vascular endothelial growth factor (VEGF) or an equivalent thereof; from about 10 ng/ml to about 100 ng/ml basic of a fibroblast growth factor (bFGF) or an equivalent thereof; from about 0.5 mM to about 5 mM of L-glutamine; from about 0.5× to about 3× of non essential amino acids; from about 50 units/ml to about 300 units/ml of penicillin; from about 50 units/ml to about 300 units/ml of streptomycin; and from about 0.01 mM to about 2 mM of 2-mercaptoethanol, admixed in a basal medium comprising from about 50% (w/w) to about 80% (w/w) of Alpha MEM and from about 20% (w/w) to about 50% (w/w) of DMEM.

In a yet further aspect, the culture medium comprises, or alternatively consists essentially of, or yet further consists of from about 1× to about 2.5× of a chemically defined serum replacement; from about 75 ng/ml to about 125 ng/ml of a VEGF or an equivalent thereof; from about 40 ng/ml to about 60 ng/ml of a bFGF or an equivalent thereof; from about 1 mM to about 3 mM of L-glutamine; from about 0.8× to about 1.5× of non essential amino acids; from about 80 units/ml to about 150 units/ml of penicillin; from about 80 units/ml to about 150 units/ml of streptomycin; and from about 0.05 mM to about 1 mM of 2-mercaptoethanol, admixed in a basal medium comprising from about 60% (w/w) to about 75% (w/w) of Alpha MEM and from about 25% (w/w) to about 40% (w/w) of DMEM.

For each of the above aspects, the amount of chemically defined serum replacement can vary among a range of values from about 0.5× to about 3×, or alternatively from 1.0× to about 3×, or alternatively 1.5× to 3×, or alternatively from about 2× to 3× or alternatively from 0.5× to 2.5×, or alternatively from 0.5× to 2×, or alternatively from 0.5× to 1.5×, or alternatively from 0.5× to lx, or alternatively from 1× to 2.5×, or alternatively from 1× to 2×, or alternatively 0.5× to 2×, or alternatively 2.5× to 3×, or alternatively less than 2.5×, or alternatively less than 2× or alternatively less than 1.5×, or alternatively less than 1×, or alternatively greater than 0.5×, or alternatively greater than 1×, or alternatively greater than 1.5×, or greater than 2×, or greater than 3×. In terms of percentage of the defined serum replacement as a portion of the complete medium, the range of values is from about 1% to about 25%, or alternatively from about 5%, or alternatively 10%, or alternatively 15%, or alternatively from about 20% to about 25%. Other non-limiting value ranges are from about 5%, or alternatively 10%, or alternatively 15%, to about 20%. Appropriate values can be empirically determined by those of skill in the art.

For each of the above aspects, the amount of a vascular endothelial growth factor (VEGF) or an equivalent thereof can vary among a range of values, from about 1 ng/ml to about 170 ng/ml, or alternatively from about 5 ng/ml to about 160 ng/ml, or alternatively from about 10 ng/ml to about 150 ng/ml, or alternatively from about 25 ng/ml to about 125 ng/ml, or alternatively from about 50 ng/ml to about 100 ng/ml; or approximately from about 1 ng/ml to about 150 ng/ml; or alternatively from about 1 ng/ml to 100 ng/ml; or alternatively from about 1 ng/ml to about 75 ng/ml; or alternatively from about 25 ng/ml to about 170 ng/ml; or alternatively from about 25 ng/ml to about 100 ng/ml.

For each of the above aspects, the amount of basic fibroblast growth factor (bFGF) or an equivalent thereof can vary among a range of values, from about 1 ng/ml to about 100 ng/ml; or alternatively from about 15 ng/ml to about 75 ng/ml; or alternatively from about 25 ng/ml to about 75 ng/ml; or alternatively from about 50 ng/ml to about 100 ng/ml; or alternatively from about 10 ng/ml to about 50 ng/ml, or alternatively about 10 ng/ml, or alternatively about 20 ng/ml, or alternatively about 30 ng/ml or alternatively about 40 ng/ml, or alternatively about 50 ng/ml, or alternatively about 60 ng/ml, or alternatively about 70 ng/ml, or alternatively about 80 ng/ml, or alternatively about 90 ng/ml, or alternatively about 100 ng/ml.

For each of the above aspects, the amount of mM L-glutamine can vary among a range of values, from about 0.5 mM to about 5 mM L-glutamine, or alternatively from about 1 mM to about 4 mM, or alternatively from about 0.5 mM to about 4 mM; or alternatively from about 0.5 mM to about 3 mM, or alternatively from about 1 mM to about 3 mM, or alternatively about 0.5 mM, or alternatively about 1 mM, or alternatively about 1.5 mM, or alternatively about 2 mM, or alternatively about 2.5 mM, or alternatively about 3 mM, or alternatively about 4 mM, or alternatively about 5 mM.

For each of the above aspects, the amount of penicillin and/or streptomycin can individually vary among a range of values, from about 50 to about 300 units/ml, or alternatively from about 50 units/ml to about 250 units/ml, or alternatively from about 100 units/ml to about 200 units/ml; or alternatively from about 50 units/ml to about 200 units/ml; or alternatively from about 50 units/ml to about 150 units/ml, or alternatively from about 100 units/ml to about 300 units/ml, or alternatively from about 150 units/ml to about 300 units/ml; or alternatively from about 200 units/ml to 300 units/ml, or alternatively about 50 units/ml, or alternatively about 75 units/ml, or about 100 units/ml, or alternatively about 150 units/ml, or alternatively about 200 units/ml, or alternatively about 250 units/ml or alternatively about 300 units/ml.

For each of the above aspects, the amount of 2-mercaptoethanol can vary among a range of values, from about 0.01 to about 2 mM, or alternatively from about 0.05 mM to about 2 mM; or alternatively from about 0.1 mM to about 2 mM; or alternatively from about 0.15 mM to about 2 mM, or alternatively 2 mM, or alternatively from about 0.5 mM to about 2 mM; or alternatively 0.01 to about 1 mM, or alternatively from about 0.05 mM to about 1 mM; or alternatively from about 0.1 mM to about 1 mM; or alternatively from about 0.15 mM to about 1 mM, or alternatively about 0.5 mM, or alternatively about 1.0 mM, or alternatively about 1.5 mM, or alternatively about 2 mM.

For each of the above aspects, the amount of Alpha MEM can vary among a range of values, from about 50% (w/w) to about 75% (w/w), or alternatively from about 60% to about 80%, or alternatively about 50%, or alternatively about 60%, or alternatively about 70%, or alternatively about 80%.

For each of the above aspect, the amount of DMEM can vary among a range of values, from about 20% (w/w) to about 50% (w/w), or alternatively from about 20% to about 40%, or alternatively from about 20% to about 30%, or alternatively from about 30% to about 50%, or alternatively from about 30% to about 40%, or alternatively about 20%, or alternatively about 30%, or alternatively about 40%, or alternatively about 50% all in (w/w).

In a further aspect, the culture media comprises, or alternatively consists essentially of, or yet further consists of, about 2× of a chemically defined serum replacement; about 100 ng/ml VEGF or an equivalent thereof; about 50 ng/ml bFGF or an equivalent thereof; about 2 mM L-glutamine; about 1× non essential amino acids; about 100 units/ml penicillin; about 100 units/ml streptomycin; and about 0.1 mM 2-mercaptoethanol, admixed in a basal medium comprising from about 70% (w/w) Alpha MEM and from about 30% DMEM.

Non-limited examples of chemically defined serum replacement is of Nutridoma CS, TCH™, KnockOut™ Serum Replacement, equivalents thereof or combinations thereof. In one particular aspect, the chemically defined serum replacement is Nutridoma CS.

The culture medium is particularly suited to culture and differentiate stem cells, e.g., adult, embryonic, iPSC, bone marrow stem cells, cord blood stem cells or parthenogenetic stem cells. The cells can be isolated from any source such as an animal or mammal as described above and can be a cultured cell from a cell line or a primary isolated from a patient or subject. In a particular aspect, the stem cells are mammalian embryonic stem cells, e.g., murine or human.

Further provided is a cell culture medium comprising, or alternatively consisting essentially of, or yet further consisting of, from about 10% (w/w) to about 30% (w/w) of a chemically defined serum replacement; from about 50 units/ml to about 300 units/ml penicillin; from about 50 units/ml to about 300 units/ml streptomycin; from about 0.5× to about 3× non essential amino acids; from about 0.5 mM to about 5 mM L-glutamine, from about 0.01 mM to about 2 mM 2-mercaptoethanol; from about 10 ng/ml to about 100 ng/ml vascular endothelial growth factor (VEGF) or an equivalent thereof; and from about 1 ng/ml to about 20 ng/ml bone morphogenetic protein 4 (BMP4) or an equivalent thereof or about 3 ng/ml to about 200, or alternatively from about 3 n/gml to about 150 ng/ml Activin A or an equivalent thereof, admixed in Alpha MEM.

In a further aspect, cell culture medium comprises, or alternatively consists essentially of, or yet further consists of, from about 15% (w/w) to about 25% (w/w) of a chemically defined serum replacement; from about 80 to about 150 units/ml penicillin; from about 80 to about 150 units/ml streptomycin; from about 0.8 to about 1.5× non essential amino acids; from about 1 to about 3 mM L-glutamine; from about 0.05 to about 1 mM 2-mercaptoethanol; from about 20 ng/ml to about 50 ng/ml vascular endothelial growth factor (VEGF) or an equivalent theroef; and from about 3 ng/ml to about 10 ng/ml bone morphogenetic protein 4 (BMP4) or an equivalent thereof or from about 3 ng/ml to about 200 ng/ml, or alternatively from about 3 ng/ml to about 150 ng/ml Activin A, admixed in Alpha MEM.

This invention also provides a cell culture medium comprising, or alternatively consisting essentially of, or yet further consisting of about 20% (w/w) of a chemically defined serum replacement; about 100 units/ml penicillin; about 100 units/ml streptomycin; about 1× non essential amino acids; about 2 mM L-glutamine; about 0.1 mM 2-mercaptoethanol; about 30 ng/ml vascular endothelial growth factor (VEGF) or an equivalent thereof; and about 5 ng/ml bone morphogenetic protein 4 (BMP4) or an equivalent thereof, admixed in Alpha MEM.

In a specific aspect, the invention provides a cell culture medium comprising, or alternatively consisting essentially of, or yet further consisting of, about 20% (w/w) of a chemically defined serum replacement; about 100 units/ml penicillin; about 100 units/ml streptomycin; about 1× non essential amino acids; about 2 mM L-glutamine; about 0.1 mM 2-mercaptoethanol; about 30 ng/ml vascular endothelial growth factor (VEGF) or an equivalent thereof; and about 100/ng/ml Activin A, admixed in Alpha MEM.

For each of the above aspects, the amount of a chemically defined serum replacement can vary among a range of values from about 10% (w/w) to about 30% (w/w); or alternatively from about 10% to about 20%; or alternatively from about 20% to 30%; or alternatively from 15% to about 30%; or alternatively from about 15% to about 20% or alternatively about 10%, or alternatively about 15%, or alternatively about 20%, or alternatively about 25%, or alternatively about 30%.

For each of the above aspects, the amount of non essential amino acids can vary among a range of values from about 0.5× to about 3×, or alternatively from about 1.0× to about 3×, or alternatively from about 1.5× to about 3×, or alternatively from about 2× to 3× or alternatively from about 0.5× to about 2.5×, or alternatively from about 0.5× to about 2×, or alternatively from about 0.5× to about 1.5×, or alternatively from about 0.5× to about 1×, or alternatively from about 1× to about 2.5×, or alternatively from about 1× to 2×, or alternatively from about 0.5× to about 2×, or alternatively from about 2.5× to about 3×, or alternatively less than about 2.5×, or alternatively less than about 2× or alternatively less than about 1.5×, or alternatively less than about 1×, or alternatively greater than about 0.5×, or alternatively greater than about 1×, or alternatively greater than about 1.5×, or greater than about 2×, or greater than about 3×. Alternatively, the amount of non essential amino acids for a general solution should be from about 0.25% to 5%, or alternatively from about 0.5%, or from about 1%, or alternatively from 1.5%, or alternatively from 2.0% to 5%. Alternative ranges also includes 0.5% to about 5%, or alternatively to 4.5%, or alternatively to 4.0%, or alternatively to 3.5%, or alternatively about 3.0%. The most effective amount can be empirically determined by one of skill in the art.

For each of the above aspects, the amount of vascular endothelial growth factor (VEGF) or an equivalent thereof can vary among a range of values, from about 1 ng/ml to about 100 ng/ml, or alternatively from about 5 ng/ml to about 100 ng/ml, or alternatively from about 10 ng/ml to about 75 ng/ml, or alternatively from about 25 ng/ml to about 75 ng/ml, or alternatively from about 50 ng/ml to about 100 ng/ml; or approximately from about 1 ng/ml to about 50 ng/ml; or alternatively from about 1 ng/ml to 40 ng/ml; or alternatively from about 10 ng/ml to about 30 ng/ml; or alternatively about 20 ng/ml; or alternatively about 30 ng/ml; or alternatively about 40 ng/ml; or alternatively about 50 ng/ml.

For each of the above aspects, the amount of bone morphogenetic protein 4 (BMP4) or an equivalent thereof basic can vary among a range of values, from about and from about 1 ng/ml to about 20 ng/ml; or alternatively from about 1 ng/ml to about 15 ng/ml; or alternatively from about 5 ng/ml to about 20 ng/ml; or alternatively from about 10 ng/ml to about 20 ng/ml; or alternatively from about 15 ng/ml to about 20 ng/ml; or alternatively from about 15 ng/ml to about 15 ng/ml, or alternatively about 1 ng/ml, or alternatively about 5 ng/ml, or alternatively about 10 ng/ml or alternatively about 15 ng/ml, or alternatively about 20 ng/ml.

For each of the above aspects, the amount of mM L-glutamine can individually vary among a range of values, from about 0.5 mM to about 5 mM L-glutamine, or alternatively from about 1 mM to about 4 mM, or alternatively from about 0.5 mM to about 4 mM; or alternatively from about 0.5 mM to about 3 mM, or alternatively from about 1 mM to about 3 mM, or alternatively about 0.5 mM, or alternatively about 1 mM, or alternatively about 1.5 mM, or alternatively about 2 mM, or alternatively about 2.5 mM, or alternatively about 3 mM, or alternatively about 4 mM, or alternatively about 5 mM.

For each of the above aspects, the amount of penicillin and streptomycin can vary among a range of values, from about 50 to about 300 units/ml, or alternatively from about 50 units/ml to about 250 units/ml, or alternatively from about 100 units/ml to about 200 units/ml; or alternatively from about 50 units/ml to about 200 units/ml; or alternatively from about 50 units/ml to about 150 units/ml, or alternatively from about 100 units/ml to about 300 units/ml, or alternatively from about 150 units/ml to about 300 units/ml; or alternatively from about 200 units/ml to 300 units/ml, or alternatively about 50 units/ml, or alternatively about 75 units/ml, or about 100 units/ml, or alternatively about 150 units/ml, or alternatively about 200 units/ml, or alternatively about 250 units/ml or alternatively about 300 units/ml.

For each of the above aspects, the amount of 2-mercaptoethanol can vary among a range of values, from about 0.01 mM to about 2 mM, or alternatively from about 0.05 mM to about 2 mM; or alternatively from about 0.1 mM to about 2 mM; or alternatively from about 0.15 mM to about 2 mM, or alternatively about 2 mM, or alternatively from about 0.5 mM to about 2 mM; or alternatively from about 0.01 to about 1 mM, or alternatively from about 0.05 mM to about 1 mM; or alternatively from about 0.1 mM to about 1 mM; or alternatively from about 0.15 mM to about 1 mM, or alternatively about 0.5 mM, or alternatively about 1.0 mM, or alternatively about 1.5 mM, or alternatively about 2 mM.

Non-limited examples of chemically defined serum replacement is of Nutridoma CS, TCH™, KnockOut™ Serum Replacement, equivalents thereof or combinations thereof. In one particular aspect, the chemically defined serum replacement is KnockOut™ Serum Replacement.

The culture medium is particularly suited to culture and differentiate stem cells, e.g., adult, embryonic, iPSC, parthenogenetic stem cells, an adult bone marrow stem cell or a cord blood stem cell. The cells can be isolated from any source such as an animal or mammal as described above and can be a cultured cell from a cell line or a primary isolated from a patient or subject. In a particular aspect, the stem cells are mammalian embryonic stem cells, e.g., murine or human.

### Cell Culture Systems

This invention also provides a cell culture system or “kit” comprising of the cell culture mediums as described above alone or in combination with each other. For example, the system is a culture medium comprising, or alternatively consisting essentially of, or yet further consisting of, from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml of a basic fibroblast growth factor (bFGF) or an equivalent thereof; from about 0.5 mM to about 5 mM of L-glutamine; from about 0.5× to about 3× of non essential amino acids; from about 50 units/ml to about 300 units/ml of penicillin; from about 50 units/ml to about 300 units/ml of streptomycin; and from about 0.01 mM to about 2 mM 2-mercaptoethanol, admixed in a basal medium comprising from about 50% to about 80% (w/w) of Alpha MEM and from about 20% (w/w) to about 50% (w/w) of DMEM. The components can vary as described above and are incorporated herein by reference. The second component is a cell culture medium comprising, or alternatively consisting essentially of, or yet further consisting of, from about 10% to about 30% (w/w) of a chemically defined serum replacement; from about 50 units/ml to about 300 units/ml of penicillin; from about 50 units/ml to about 300 units/ml of streptomycin; from about 0.5× to about 3× of non essential amino acids; from about 0.5 mM to about 5 mM of L-glutamine, from about 0.01 mM to about 2 mM of 2-mercaptoethanol; from about 10 ng/ml to about 100 ng/ml vascular endothelial growth factor (VEGF) or an equivalent thereof; and from about 1 ng/ml to about 20 ng/ml of bone morphogenetic protein 4 (BMP4) or an equivalent thereof or about 3 ng/ml to about 200 ng/ml or alternatively about 3 ng/ml to about 150 ng/ml of Activin A or an equivalent thereof, admixed in of Alpha MEM. The components can vary as described above and are incorporated herein by reference.

In a further aspect, the cell culture system of further comprises, or alternatively consists essentially of or yet further consists of a cell culture container and instructions for culturing and differentiating the cells. A non-limited example is a cell culture plate such as a microwell plate. The plates can be coated to further facilitate differentiation.

Further provided is a method for culturing one or more isolated stem cells by contacting and culturing the one or more stem cells in a cell culture medium of this invention. The culture medium is particularly suited to culture and differentiate stem cells, e.g., adult, embryonic, iPSC, parthenogenetic stem cells, an adult bone marrow stem cell or a cord blood stem cell. The cells can be isolated from any source such as an animal or mammal as described above and can be a cultured cell from a cell line or a primary isolated from a patient or subject. In a particular aspect, the stem cells are mammalian embryonic stem cells, e.g., murine or human.

In one aspect, the invention provides a method for differentiating an isolated stem cell, comprising incubating the isolated stem cell in a first cell culture medium comprising, or alternatively consisting essentially of, or yet further consisting of, from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml of a basic fibroblast growth factor (bFGF) or an equivalent thereof; from about 0.5 mM to about 5 mM of L-glutamine; from about 0.5× to about 3× of non essential amino acids; from about 50 units/ml to about 300 units/ml of penicillin; from about 50 units/ml to about 300 units/ml of streptomycin; and from about 0.01 mM to about 2 mM of 2-mercaptoethanol, admixed in a basal medium comprising from about 50% to about 80% (w/w) of Alpha MEM and from about 20% (w/w) to about 50% (w/w) of DMEM. The components can vary as described above and are incorporated herein by reference. Thereafter, the cells are cultured in a second cell culture medium comprising, or alternatively consisting essentially of, or yet further consisting of, from about 10% to about 30% (w/w) of a chemically defined serum replacement; from about 50 units/ml to about 300 units/ml of penicillin; from about 50 units/ml to about 300 units/ml of streptomycin; from about 0.5× to about 3× of non essential amino acids; from about 0.5 mM to about 5 mM of L-glutamine, from about 0.01 mM to about 2 mM of 2-mercaptoethanol; from about 10 ng/ml to about 100 ng/ml of a vascular endothelial growth factor (VEGF) or an equivalent thereof; and from about 1 ng/ml to about 20 ng/ml of a bone morphogenetic protein 4 (BMP4) or an equivalent thereof or about 3 ng/ml to about 200 ng/ml or alternatively about 3 ng/ml to about 150 ng/ml of Activin A or an equivalent thereof, admixed in Alpha MEM. The components can vary as described above and are incorporated herein by reference.

The method is particularly suited to culture and differentiate stem cells, e.g., adult, embryonic, iPSC, parthenogenetic stem cells, an adult bone marrow stem cell or a cord blood stem cell. The cells can be isolated from any source such as an animal or mammal as described above and can be a cultured cell from a cell line or a primary isolated from a patient or subject. In a particular aspect, the stem cells are mammalian embryonic stem cells, e.g., murine or human. The method is particularly useful for preparing a population of endothelial cells, e.g., arterial or vascular endothelial cells. The cells can be isolated from the culture medium using well known methods such as centrifugation. The populations are substantially homogenous.

The cells and populations can be used for research or therapeutically, and therefore can be allogeneic or autologous to the subject being treated. The can be further modified by insertion of an exogenous polynucleotide or polypeptide using known methods. They can further be combined with a pharmaceutically acceptable carrier. Thus, this invention also provides a method for treating a subject, comprising, or alternatively consisting essentially of, or yet further consisting of, administering a population of cells prepared by the methods of this invention to the subject thereby treating the subject. The cells can be allogeneic or autologous to the subject. They are useful to treat a subject is suffering from a condition of one or more of peripheral artery disease, ischemic heart disease, or cerebral ischemia.

### Screens and Assays

This invention also provides a method for identifying an agent that modulates stem cell differentiating comprising, or alternatively consisting essentially of or yet further consists of, contacting the agent with a stem cell and a cell culture medium under conditions that favor cell growth and/or differentiation, and assaying for stem cell differentiation. The cell culture medium comprises, or alternatively consists essentially of, or yet further consists of, from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml of a basic fibroblast growth factor (bFGF) or an equivalent thereof; from about 0.5 to about 5 mM of L-glutamine; from about 0.5× to about 3× of non essential amino acids; from about 50 to about 300 units/ml of penicillin; from about 50 to about 300 units/ml of streptomycin; and from about 0.01 to about 2 mM of 2-mercaptoethanol, admixed in a basal medium comprising from about 50% to about 80% (w/w) of Alpha MEM and from about 20% to about 50% of DMEM. Alternatively, one or more of the above components can be omitted and the agent can be tested for its equivalence to the omitted element.

This invention also provides a method for identifying an agent that modulates stem cell differentiating comprising, or alternatively consisting essentially of, or yet further consists of, contacting the agent with a stem cell and a cell culture medium under conditions that favor cell growth and/or differentiation, and assaying for stem cell differentiation. The cell culture medium comprises, or alternatively consists essentially of, or yet further consists of, from about 10% (w/w) to about 30% (w/w) of a chemically defined serum replacement; from about 50 units/ml to about 300 units/ml of penicillin; from about 50 units/ml to about 300 units/ml of streptomycin; from about 0.5× to about 3× of non essential amino acids; from about 0.5 mM to about 5 mM of L-glutamine; from about 0.01 mM to about 2 mM of 2-mercaptoethanol; from about 10 ng/ml to about 100 ng/ml of a vascular endothelial growth factor (VEGF) or an equivalent thereof; and from about 1 ng/ml to about 20 ng/ml of a bone morphogenetic protein 4 (BMP4) or an equivalent thereof or about 3 ng/ml to about 200 ng/ml of Activin or an equivalent thereof; admixed in Alpha MEM. Alternatively, one or more of the above components can be omitted and the agent can be tested for its equivalence to the omitted element.

Further provided is a method for identifying an agent that modulates stem cell differentiating comprising, or alternatively consisting essentially of, or yet further consisting of, contacting the agent with a stem cell and a stem cell medium, wherein the medium comprises, or alternatively consists essentially of, or yet further consists of, from about 10% (w/w) to about 30% (w/w) of a chemically defined serum replacement; from about 50 units/ml to about 300 units/ml of penicillin; from about 50 units/ml to about 300 units/ml of streptomycin; from about 0.5× to about 3× of non essential amino acids; from about 0.5 mM to about 5 mM of L-glutamine; from about 0.01 mM to about 2 mM of 2-mercaptoethanol; from about 10 ng/ml to about 100 ng/ml of a vascular endothelial growth factor (VEGF) or an equivalent thereof; and from about 1 ng/ml to about 20 ng/ml of a bone morphogenetic protein 4 (BMP4) or an equivalent thereof or about 3 ng/ml to about 200 ng/ml of Activin or an equivalent thereof, admixed in of Alpha MEM and then, subsequently contacting the cells with a cell culture medium that comprises, or alternatively consists essentially of, or yet further consists of, from about from about 0.5× to about 3× of a chemically defined serum replacement; from about 1 ng/ml to about 170 ng/ml of a vascular endothelial growth factor (VEGF) or an equivalent thereof; from about 1 ng/ml to about 100 ng/ml of a basic fibroblast growth factor (bFGF) or an equivalent thereof; from about 0.5 to about 5 mM of L-glutamine; from about 0.5× to about 3× of non essential amino acids; from about 50 to about 300 units/ml of penicillin; from about 50 to about 300 units/ml of streptomycin; and from about 0.01 to about 2 of mM 2-mercaptoethanol, admixed in a basal medium comprising from about 50% to about 80% (w/w) of Alpha MEM and from about 20% to about 50% of DMEM, under conditions that favor cell growth and/or differentiation, and assaying for stem cell differentiation. The subsequent medium can replace or supplement the first added cell culture medium. Alternatively, one or more of the above components can be omitted and the agent can be tested for its equivalence to the omitted element.

The cells for these screens include an isolated embryonic stem cell, an IPSC, adult bone marrow stem cell, a cord blood stem cell, an parthenogenetic stem cell or an isolated embryoid body. In some aspects, they are primary cells or they can be cultured stem cells.

For the purposes of this invention, an “agent” is intended to include, but not be limited to a biological or chemical compound such as a simple or complex organic or inorganic molecule, a peptide, a protein (e.g. antibody), a polynucleotide (e.g. anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One preferred embodiment is a method for screening small molecules capable of interacting with the protein or polynucleotide of the invention. For the purpose of this invention, “small molecules” are molecules having low molecular weights (MW) that are, in one embodiment, capable of binding to a protein of interest thereby altering the function of the protein. Preferably, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell culture or tissue are first provided. The cells are cultured under conditions (temperature, growth or culture medium and gas (CO₂)) and for an appropriate amount of time to attain exponential proliferation without density dependent constraints. It also is desirable to maintain an additional separate cell culture that is not infected as a control.

As is apparent to one of skill in the art, suitable cells can be cultured in micro-titer plates and several agents can be assayed at the same time by noting genotypic changes, phenotypic changes or a reduction in microbial titer.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined.

### Experimental Examples

The present technology is further understood by reference to the following example. The present technology is not limited in scope by the examples, which are intended as illustrations of aspects of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims.

### Stem Cell Source

Applicants set out to explore and develop differentiation methodology under chemically defined conditions for the generation of highly pure and functional endothelial cells from embryonic stem cells. Here, Applicants present the results of these investigations regarding optimal time points required for the generation of high numbers of vascular progenitor cells, optimal substrate(s), and several chemically defined medium formulations for cells at various stages of maturation. The methods presented in this application using chemically defined medium will consistently generate endothelial cells with appropriate expression of several endothelial markers with better LDL-uptake compared with previous methods using serum [18].

### Materials and Methods

### Murine Embryonic Stem Cell Culture

R1 murine embryonic stem cells (ATTC) were cultured on 0.5% gelatin coated-cell culture dishes as previously described in Blancas, et al., Current Protocols In Stem Cell Biology, 2008, except in a new serum-free medium. This medium contains KnockOut DMEM (Invitrogen), KnockOut Serum Replacement (Invitrogen), Penicillin-Streptomycin (Invitrogen), Non essential Amino Acids (Invitrogen), L-glutamine (Invitrogen), 2-mercaptoethanol (Calbiochem), Leukemia Inhibitory Factor (ESGRO; Chemicon), and Bone Morphogenetic Protein 4 (R&D Systems).

### Derivation to Flk-1+ Cells in Chemically Defined Medium

Undifferentiated R1 mESC were harvested from gelatin-coated dishes using 0.25% Trypsin/2.21 mM EDTA (Mediatech) and plated on cell culture plates coated with various commercially available substrates (BD Biosciences). Initial induction medium (called NS1D2b), consisted of Alpha-MEM (Cellgro), 20% KnockOut Serum Replacement (Invitrogen), 1× Penicillin-Streptomycin (Invitrogen), 1× Non essential Amino Acids (Invitrogen), 2 mM L-glutamine (Invitrogen), 0.1 mM 2-mercaptoethanol (Calbiochem), 30 ng/ml Vascular Endothelial Growth Factor (R&D Systems) and 5 ng/ml Bone Morphogenetic Protein 4 (R&D Systems). Because induction times for generating vascular progenitor cells (Flk-1+ cells) does vary between cell lines [23], undifferentiated ESC were first cultured on plates in NS1D2b medium for 2, 3, 4 and 5 days in order to determine the optimal number of days for initial induction of Flk-1+ vascular progenitor cells.

In addition to verifying induction time, Applicants also determined the most appropriate substrate for induction by using culture plates coated in either 0.5% gelatin, 50 μg/ml fibronectin, 50 μg/ml collagen type I, 50 μg/ml collagen type IV, and 50 μg/ml laminin as per manufacturer instructions (BD Biosciences). Adherent cells were counted and analyzed via flow cytometry for Flk-1 expression using AlexaFluor 647 conjugated anti-mouse anti-Flk-1 antibodies (Biolegend).

### Generating Endothelial Cells in Chemically Defined Conditions

After initial induction period, the cell population was enriched for Flk-1+ vascular progenitor cells using a MiniMACS (Miltenyi Biotec). AlexaFluor 647 conjugated anti-mouse anti-Flk-1 antibodies (Biolegend) and anti-AlexaFluor 647 magnetic beads (Miltenyi Biotec) were used to label the Flk-1+ expressing cells. Post enrichment, the Flk-1+ cells were replated on plates coated in either fibronectin, laminin, collagen type-I, collage type-IV, or gelatin, allowed to grow up for at least 7 days, and fed with a medium developed in Applicants' lab called LDSk, the medium as described above. The medium begins with basal mixture of 70% Alpha DMEM (Mediatech) and 30% DMEM (Invitrogen) to which is added 2× Nutridoma CS, 100 ng/ml VEGF, 50 ng/ml bFGF, 2 mM L-glutamine, 1× Non Essential Amino Acids, 1× Penicillin-Streptomycin (100 units/ml of Penicillin and 100 ug/ml Streptomycin), and 0.1 mM 2-mercaptoethanol. Cells were passed 1-4 times until clear cobblestone morphology became visible.

### Second Purification of EC

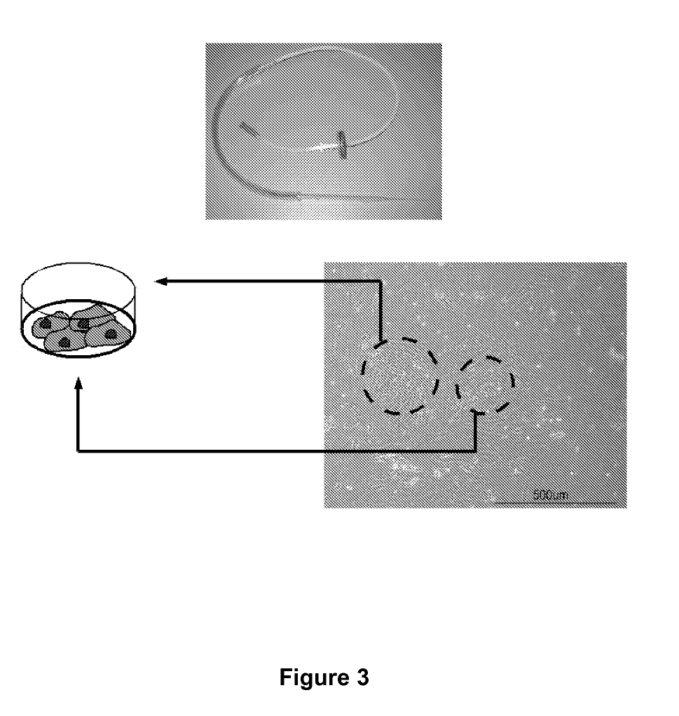

Endothelial cells with cobblestone morphology were manually picked with flame-pulled micro-tip Pasteur pipettes in a sterile laminar flow hood outfitted with a stereoscope (Zeiss). The 9″ Pasteur pipettes (VWR) were flame-pulled to a thin point and attached to a mouth aspirator line (Sigma-Aldrich) with a 0.22 μg/ml filter (Whatman) for performing the sterile manual selection [23]. The culture plates were washed with phosphate buffered saline followed by incubation with Cell Dissociation Buffer for 10 minutes (Invitrogen) in order to allow gentle cell scraping and cell aspiration with the micro-tip pipette (FIG. 3). The manually picked cells were then plated onto fibronectin-coated dishes in LDSk medium.

### Flow Cytometric Analysis

Differentiated EC were stained for the following endothelial markers: Flk-1 (Biolegend), vascular endothelial (VE) cadherin (eBioscience), Flt-1 (Santa Cruz), EphB4 (Santa Cruz), ephrin-B2 (Santa Cruz), and Tie-1 (Santa Cruz). All samples were analyzed using a BD LSRII flow cytometer and FlowJo software (TreeStar).

### LDL-Uptake

EC derived under chemically defined conditions were plated on Permanox microscope slides (NUNC). Commercially available Alexa Fluor 488 Acetylated-LDL (Invitrogen) was diluted to 1:100 in DMEM (Invitrogen) and incubated with the cells for 4 hours at 37° C. The slides were then stained with DAPI and fixed with 4% formaldehyde. The slides were imaged with a Leica fluorescent scope.

### Results

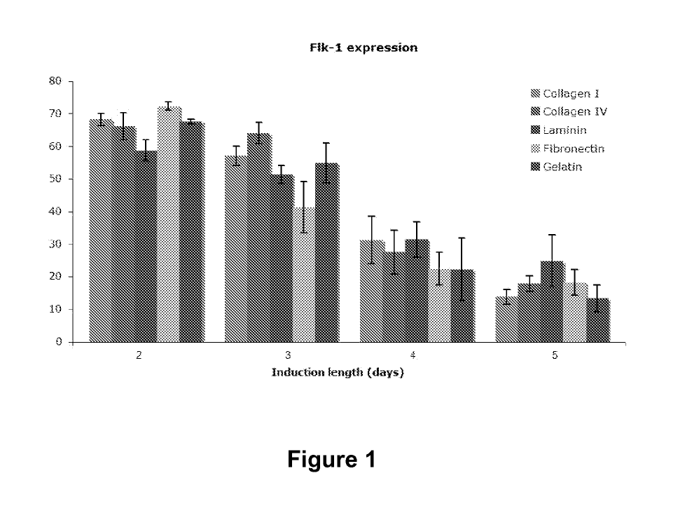

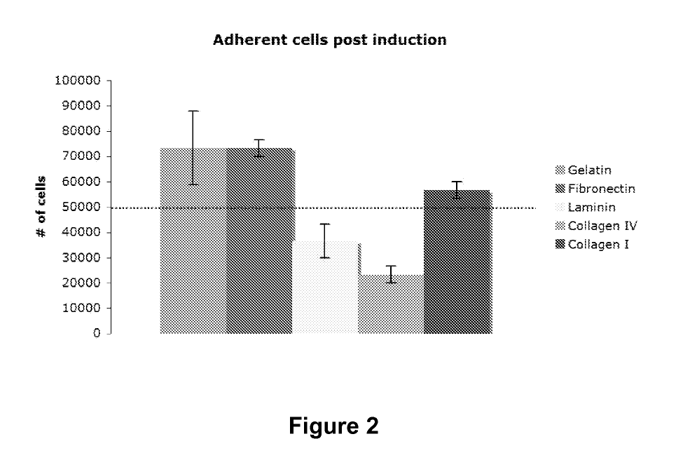

Undifferentiated mESC were cultured on the substrate-coated plates in NS1D2b medium for 2, 3, 4 and 5 days—limited cell adhesion and differentiation prevented the testing of a timepoint at day 1. For R1 ESC, an induction time of 2 days yields the greatest number of Flk-1+ cells (FIG. 1). Applicants also note that the Flk-1 expression levels were similar on several substrates at Day 2, and none were statistically better than the others. Applicants also counted the total number of adherent cells at the end of the 2 day induction period. The data shows that induction on fibronectin yields the optimal number of Flk-1 cells, (FIG. 2). Although many studies recommend the use of collagen type IV [14] as the differentiation substrate, here Applicants show that in the absence of serum, yield of Flk-1+ cells on this substrate was relatively low.

Here Applicants present characterization data from two sets of mESC-derived EC (dEC-3-13 and dEC-3-11) generated independently using chemically defined mediums. Both sets of ESC-derived EC exhibited cobblestone morphologies and grew into well-defined endothelial sheets facilitating easy manual selection further purification of the endothelial cells. After this second purification step, the cells were expanded and maintained in LDSk medium. Both groups of EC robustly expressed the endothelial markers Flk-1, VE-cadherin, Flt-1, EphB4, and Tie-1. In chemically defined culture, the expression of the arterial maker ephrin-B2 is also noted, although its expression is greater in one batch than the other batch of ESC-derived EC.

The documented presence of arterial cells in the chemically-defined culture introduces an additional possibility of isolating and expanding these cells for possible therapeutic uses.

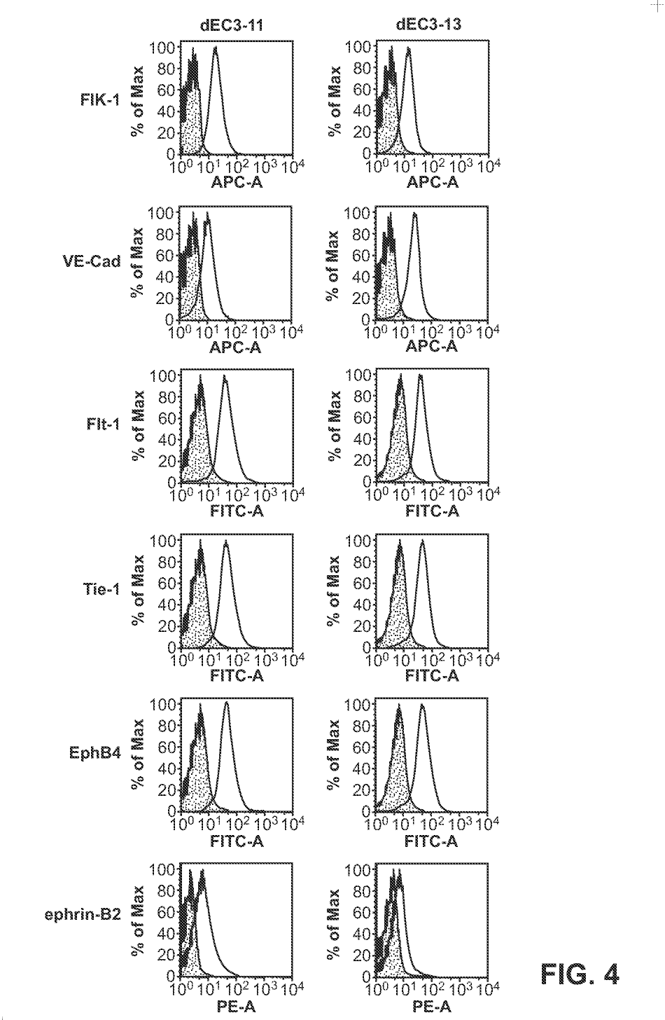

These cells also exhibit similar EC marker expression profiles as an ESC-derived EC derived in serum containing conditions previously described (FIG. 4) [12, 18].

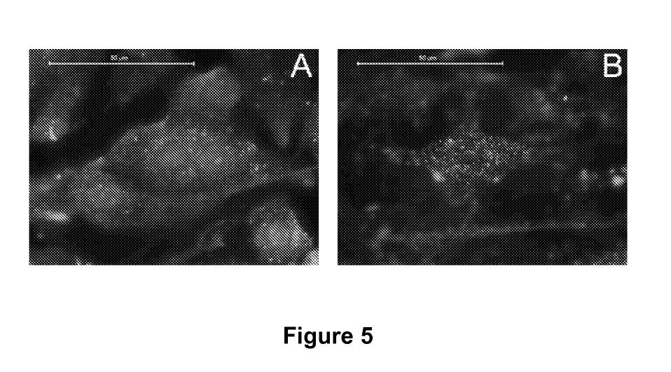

The uptake of low-density lipoproteins is a hallmark of proper arterial EC function, and is therefore an important aspect to investigate to determine if the EC derived in chemically defined culture is indeed functional. As seen in FIG. 5, virtually all EC derived in chemically defined conditions uptake LDL. This is an essential function that EC previously derived in serum containing conditions lacked [12, 18].

### Discussion

The possibility of generating EC in chemically defined conditions holds tremendous potential for therapeutic applications. In addition to eliminating various unknown animal contaminants, chemically defined culture conditions facilitate the study of the effects of growth factors and other cytokines by removing unspecified elements found in serum. By creating a chemically defined differentiation condition, the reproducibility of EC derivation increases, another key consideration for therapeutic applications.

The initial stage of induction requires the addition of factors that promote the differentiation of cells into the desired population. Flk-1, also called VEGF receptor 2, is considered to be the first lineage commitment marker for cells that will become vascular progenitors or hematopoietic progenitors [14, 24, 25]. Induction of Flk-1 expression in ESC, for the purpose of studying the process of vascular development, in serum free medium has led to the investigation of angiogenic factors and factors that promote mesoderm formation [1, 4, 9, 26].

VEGF binds to Flk-1 which promotes EC survival by activating the PI3 kinase/Akt pathway ultimately leading to the inhibition of caspase activity. Although Flk-1 is not expressed by SMC, they respond to VEGF stimulation. Specifically, the binding of VEGF to VEGF receptor 1 (fms-like tyrosine kinase 1 Flt-1), causes the upregulation of matrix metalloproteinase-9 [27]. This facilitates the degradation of the extracellular matrix during angiogenesis [28].

Basic fibroblast growth factor (bFGF) is also considered to be an angiogenic factor. However, it did not increase mesoderm formation under serum free conditions [29]. Alternatively, Bone Morphogenetic Protein 4 (BMP-4) and Activin A (RDSystems) succeed in promoting mesodermal specific gene expression in serum free differentiation conditions. BMP-4, in concert with VEGF, is used in Applicants' current serum free differentiation medium since it promotes ventral mesoderm and hematopoietic development and inhibits neuronal development [29-31].

Applicants have shown that the EC generated in their lab not only express several endothelial-specific markers, but also maintain a level of functionality previously undetected in EC derived from mESC in previous studies [18]. The importance of generating functional EC in vitro reproducibly in serum free culture benefits not only cardiovascular research, but also research into the differentiation mechanisms of the other cell lines of interest.

It has been observed that the venous lineage is the default pathway of EC differentiation in static conditions [22]. Applicants' flow cytometry data on their derived EC further confirms this finding. Other studies have shown that shear stress can affect the lineage selection of EC, favoring the arterial lineage. By exposing Applicants' derived EC to various levels of shear stress, Applicants can attempt to further control differentiation, thus generating easily reproducible methods of differentiating lineage specific EC in vitro in chemically defined conditions. The proposed experimental range of shear stress is 0.1-40 dynes/cm². Arterial levels of shear stress are 15 dynes/cm² and above, while the venous range is below 10 dynes/cm^{2 [}32, 33].

### CONCLUSION

Applicants' results indicate that EC can be successfully generated in chemically defined conditions that exhibit comparable surface marker expression to EC derived in traditional serum-containing conditions. The LDL-uptake also imply that EC derived in Applicants' chemically defined conditions may be more functionally mature, as evidenced by a significantly higher uptake of LDL. Applicants believe their method of chemically defined derivation yields not only more reproducibility, which is important for developing clinically relevant applications, but also opens up possibilities for future studies on the influences of specific growth factors on EC maturity and functionality.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

### REFERENCES

1. Nishikawa, S., L. M. Jakt, and T. Era, Embryonic stem-cell culture as a tool for developmental cell biology. Nat Rev Mol Cell Biol, 2007. 8(6): p. 502-7.
2. Ishikawa, M. and T. Asahara, Endothelial progenitor cell culture for vascular regeneration. Stem Cells Dev, 2004. 13(4): p. 344-9.
3. Ishii, O., et al., In vitro tissue engineering of a cardiac graft using a degradable scaffold with an extracellular matrix-like topography. J Thorac Cardiovasc Surg, 2005. 130(5): p. 1358-63.
4. Asahara, T., et al., Isolation of putative progenitor endothelial cells for angiogenesis. Science, 1997. 275(5302): p. 964-7.
5. Laschke, M. W., et al., Angiogenesis in tissue engineering: breathing life into constructed tissue substitutes. Tissue Eng, 2006. 12(8): p. 2093-104.
6. Tremblay, P. L., et al., Inosculation of tissue-engineered capillaries with the host's vasculature in a reconstructed skin transplanted on mice. Am J Transplant, 2005. 5(5): p. 1002-10.
7. Badorff, C., et al., Transdifferentiation of blood-derived human adult endothelial progenitor cells into functionally active cardiomyocytes. Circulation, 2003. 107(7): p. 1024-32.
8. Smith, A. G., Culture and differentiation of embryonic stem cells. Methods in Cell Science, 1991. 13(2): p. 89-94.
9. Levenberg, S., et al., Endothelial cells derived from human embryonic stem cells. Proc Natl Acad Sci USA, 2002. 99(7): p. 4391-6.
10. Hirashima, M., et al., Maturation of embryonic stem cells into endothelial cells in an in vitro model of vasculogenesis. Blood, 1999. 93(4): p. 1253-63.
11. Nishikawa, S. I., et al., Cell biology of vascular endothelial cells. Ann N Y Acad Sci, 2001. 947: p. 35-40; discussion 41.
12. McCloskey, K. E., S. L. Stice, and R. M. Nerem, In vitro derivation and expansion of endothelial cells from embryonic stem cells. Methods Mol Biol, 2006. 330: p. 287-301.
13. McCloskey, K. E., M. E. Gilroy, and R. M. Nerem, Use of embryonic stem cell-derived endothelial cells as a cell source to generate vessel structures in vitro. Tissue Eng, 2005. 11(3-4): p. 497-505.
14. Nishikawa, S. I., et al., Progressive lineage analysis by cell sorting and culture identifies FLK1+VE-cadherin+ cells at a diverging point of endothelial and hemopoietic lineages. Development, 1998. 125(9): p. 1747-57.
15. Adelman, C. A., S. Chattopadhyay, and J. J. Bieker, The BMP/BMPR/Smad pathway directs expression of the erythroid-specific EKLF and GATA1 transcription factors during embryoid body differentiation in serum free media. Development, 2002. 129(2): p. 539-49.
16. Cheng, J., et al., Improved generation of C57BL/6J mouse embryonic stem cells in a defined serum free media. Genesis, 2004. 39(2): p. 100-4.
17. Fume, M., et al., Leukemia inhibitory factor as an anti-apoptotic mitogen for pluripotent mouse embryonic stem cells in a serum-free medium without feeder cells. In Vitro Cell Dev Biol Anim, 2005. 41(1-2): p. 19-28.
18. McCloskey, K. E., et al., Embryonic stem cell-derived endothelial cells may lack complete functional maturation in vitro. J Vasc Res, 2006. 43(5): p. 411-21.
19. Laflamme, M. A., et al., Cardiomyocytes derived from human embryonic stem cells in pro-survival factors enhance function of infarcted rat hearts. Nature biotechnology, 2007. 25(9): p. 1015-1024.
20. Yang, L., et al., Human cardiovascular progenitor cells develop from a KDR+ embryonic-stem-cell-derived population. Nature, 2008. 453(7194): p. 524-528.
21. Yao, S., et al. Long-term self-renewal and directed differentiation of human embryonic stem cells in chemically defined conditions. 2006: National Acad Sciences.
22. Yamashita, J. K., Differentiation of arterial, venous, and lymphatic endothelial cells from vascular progenitors. Trends Cardiovasc Med, 2007. 17(2): p. 59-63.
23. Blancas, A. A., N. E. Lauer, and K. E. McCloskey, Endothelial Differentiation of Embryonic Stem Cells. Current protocols in stem cell biology, 2008.
24. Shalaby, F., et al., Failure of blood-island formation and vasculogenesis in Flk-1-deficient mice. Nature, 1995. 376(6535): p. 62-6.
25. Dumont, D. J., et al., Vascularization of the mouse embryo: a study of flk-1, tek, tie, and vascular endothelial growth factor expression during development. Dev Dyn, 1995. 203(1): p. 80-92.
26. Olsson, A. K., et al., VEGF receptor signalling—in control of vascular function. Nat Rev Mol Cell Biol, 2006. 7(5): p. 359-71.
27. Wang, H. and J. A. Keiser, Vascular endothelial growth factor upregulates the expression of matrix metalloproteinases in vascular smooth muscle cells: role of flt-1. Circ Res, 1998. 83(8): p. 832-40.
28. Hangai, M., et al., Matrix metalloproteinase-9-dependent exposure of a cryptic migratory control site in collagen is required before retinal angiogenesis. Am J Pathol, 2002. 161(4): p. 1429-37.
29. Johansson, B. M. and M. V. Wiles, Evidence for involvement of activin A and bone morphogenetic protein 4 in mammalian mesoderm and hematopoietic development. Mol Cell Biol, 1995. 15(1): p. 141-51.
30. Stpyridis, M. P. and A. G. Smith, Neural differentiation of mouse embryonic stem cells. Biochem Soc Trans, 2003. 31(Pt 1): p. 45-9.
31. Karlsson, G., et al., Smad4 is critical for self-renewal of hematopoietic stem cells. J Exp Med, 2007. 204(3): p. 467-74.
32. Malek, A. M., S. L. Alper, and S. Izumo, Hemodynamic shear stress and its role in atherosclerosis. JAMA, 1999. 282(21): p. 2035-42.
33. Lawrence, M. B., et al., Effect of venous shear stress on CD18-mediated neutrophil adhesion to cultured endothelium. Blood, 1990. 75(1): p. 227.

What is claimed is:

1. A cell culture medium comprising:
from about 0.5× to about 3× of a chemically defined serum replacement;
from about 1 ng/ml to about 170 ng/ml of a vascular endothelial growth factor (VEGF) or an equivalent thereof;
from about 1 ng/ml to about 100 ng/ml of a basic fibroblast growth factor (bFGF) or an equivalent thereof;
from about 0.5 mM to about 5 mM L-glutamine;
from about 0.5× to about 3× of non essential amino acids;
from about 50 units/ml to about 300 units/ml penicillin;
from about 50 units/ml to about 300 units/ml streptomycin; and
from about 0.01 mM to about 2 mM 2-mercaptoethanol, admixed in a basal medium comprising from about 50% to about 80% (w/w) Alpha MEM and from about 20% to about 50% DMEM.

2. A cell culture medium comprising:
from about 0.5× to about 3× of a chemically defined serum replacement (please convert to ng/ml);
from about 10 ng/ml to about 170 ng/ml of a vascular endothelial growth factor (VEGF) or an equivalent thereof;
from about 10 ng/ml to about 100 ng/ml of a basic fibroblast growth factor (bFGF) or an equivalent thereof;
from about 0.5 mM to about 5 mM L-glutamine;
from about 0.5× to about 3× non essential amino acids;
from about 50 units/ml to about 300 units/ml penicillin;
from about 50 units/ml to about 300 units/ml streptomycin; and
from about 0.01 mM to about 2 mM 2-mercaptoethanol, admixed in a basal medium comprising from about 50% to about 80% (w/w) Alpha MEM and from about 20% to about 50% DMEM.

3. The cell culture medium of claim 1 or 2, comprising:
from about 1× to about 2.5× of a chemically defined serum replacement;
from about 75 ng/ml to about 125 of a ng/ml VEGF or an equivalent thereof;
from about 40 ng/ml to about 60 ng/ml of a bFGF or an equivalent thereof;
from about 1 mM to about 3 mM L-glutamine;
from about 0.8× to about 1.5× non essential amino acids;
from about 80 units/ml to about 150 units/ml penicillin;
from about 80 units/ml to about 150 units/ml streptomycin; and
from about 0.05 mM to about 1 mM 2-mercaptoethanol, admixed in a basal medium comprising from about 60% (w/w) to about 75% (w/w) Alpha MEM and from about 25% to about 40% DMEM.

4. The cell culture medium of claim 1 or 2 comprising:
about 2× of a chemically defined serum replacement;
about 100 ng/ml of a VEGF or an equivalent thereof;
about 50 ng/ml of a bFGF or an equivalent thereof;
about 2 mM L-glutamine;
about 1× of non essential amino acids;
about 100 units/ml penicillin;
about 100 units/ml streptomycin; and
about 0.1 mM 2-mercaptoethanol, admixed in a basal medium comprising from about 70% (w/w) Alpha MEM and from about 30% DMEM.

5. The cell culture medium of claim 1 or 2, wherein the chemically defined serum replacement is one or more of Nutridoma CS, TCH™, KnockOut™ Serum Replacement, equivalents thereof or combinations thereof.

6. The cell culture medium of claim 1 or 2, wherein the chemically defined serum replacement is Nutridoma CS.

7. A cell culture medium comprising:
from about 10% (w/w) to about 30% (w/w) of a chemically defined serum replacement;
from about 50 units/ml to about 300 units/ml penicillin;
from about 50 units/ml to about 300 units/ml streptomycin;
from about 0.5× to about 3× of non essential amino acids;
from about 0.5 mM to about 5 mM L-glutamine;
from about 0.01 mM to about 2 mM 2-mercaptoethanol;
from about 10 ng/ml to about 100 ng/ml of a vascular endothelial growth factor (VEGF) or an equivalent thereof; and
from about 1 ng/ml to about 20 ng/ml of a bone morphogenetic protein 4 (BMP4) or an equivalent thereof or about 3 ng/ml to about 200 ng/ml Activin or an equivalent thereof, admixed in Alpha MEM.

8. The cell culture medium of claim 7, comprising:
from about 15% to about 25% (w/w) of a chemically defined serum replacement;
from about 80 units/ml to about 150 units/ml penicillin;
from about 80 units/ml to about 150 units/ml streptomycin;
from about 0.8× to about 1.5× of non essential amino acids;
from about 1 mM to about 3 mM L-glutamine;
from about 0.05 mM to about 1 mM 2-mercaptoethanol;
from about 20 ng/ml to about 50 ng/ml of a vascular endothelial growth factor (VEGF) or an equivalent thereof; and
from about 3 ng/ml to about 10 ng/ml of a bone morphogenetic protein 4 (BMP4) or an equivalent thereof or from about 3 ng/ml to about 200 ng/ml Activin A, admixed in Alpha MEM.

9. A cell culture medium of claim 7, comprising:
about 20% (w/w) of a chemically defined serum replacement;
about 100 units/ml penicillin;
about 100 units/ml streptomycin;
about 1× non essential amino acids;
about 2 mM L-glutamine;
about 0.1 mM 2-mercaptoethanol;
about 30 ng/ml of a vascular endothelial growth factor (VEGF) or an equivalent thereof; and
about 5 ng/ml of a bone morphogenetic protein 4 (BMP4) or an equivalent thereof, admixed in Alpha MEM.

10. A cell culture medium of claim 7, comprising:
about 20% (w/w) of a chemically defined serum replacement;
about 100 units/ml penicillin;
about 100 units/ml streptomycin;
about 1× of non essential amino acids;
about 2 mM L-glutamine;
about 0.1 mM 2-mercaptoethanol;
about 30 ng/ml of a vascular endothelial growth factor (VEGF) or an equivalent thereof; and
about 100 ng/ml Activin A, admixed in Alpha MEM.

11. The cell culture medium of claim 7, wherein the chemically defined serum replacement is one or more of Nutridoma CS, TCH™, KnockOut™ Serum Replacement, equivalents thereof or combinations thereof.

12. The cell culture medium of claim 7, wherein the chemically defined serum replacement is KnockOut™ Serum Replacement.

13. A cell culture system comprising a cell culture medium of any one of claims 1, 2, and 7.

14. The cell culture system of claim 13, further comprising a cell culture container and instructions for use.

15. The cell culture system of claim 14, wherein the cell culture container comprises a microwell plate.

16. A method for culturing one or more isolated stem cells comprising incubating the one or more stem cells in a cell culture medium of claim 1 or 2.

17. A method for culturing one or more isolated stem cells comprising incubating the one or more stem cells in a cell culture medium of claim 7.

18. A method for differentiating an isolated stem cells, comprising incubating the one or more isolated stem cells in a first cell culture medium of claim 7 for an effective amount of time and replacing the first cell culture media with a second cell culture medium of claim 1 or 2 for an effective amount of time.

19. The method of claim 18, wherein the isolated stem cell is selected from an embryonic stem cell, an induced pluripotent stem cell, a parthenogenetic stem cell, an adult bone marrow stem cell or a cord blood stem cell.

20. The method of claim 18, wherein the isolated stem cell is an embryonic stem cell.

21. A method for preparing a population of endothelial cells, comprising:
1) incubating an embryonic stem cells in a cell culture medium of claim 7 for an effective amount of time; and
2) replacing the culture medium of step 1) with a cell culture medium of claim 1 or 2 for an effective amount of time, thereby preparing a population of endothelial cells.

22. The method of claim 21, further comprising isolating the population of endothelial cells from the cell culture medium.

23. A substantially pure population of endothelial cells prepared by a method of claim 21.

24. A substantially pure population of endothelial cells prepared by a method of claim 22.

25. The population of claim 23, wherein the endothelial cells comprise an exogenous polynucleotide or polypeptide.

26. The population of claim 24, wherein the endothelial cells comprise an exogenous polynucleotide or polypeptide.

27. A composition comprising the population of claim 23 and a carrier.

28. A composition comprising the population of claim 24 and a carrier.

29. A method for treating a subject, comprising administering a population of endothelial cells of claim 23 to the subject thereby treating the subject.

30. A method for treating a subject, comprising administering a population of endothelial cells of claim 24 to the subject thereby treating the subject.

31. A kit comprising a cell culture medium of any one of claims 1, 2 and 7 and instructions for use.

32. The kit of claim 30, further comprising one or more of a cell culture container and instructions for culturing and differentiating the cells.

33. A method for identifying an agent that modulates stem cell differentiating comprising contacting the agent with a stem cell and a cell culture medium of claim 1 or 2 under conditions that favor cell growth and/or differentiation, and assaying for stem cell differentiation.

34. A method for identifying an agent that modulates stem cell differentiating comprising contacting the agent with a stem cell and a cell culture medium of claim 7 under conditions that favor cell growth and/or differentiation, and assaying for stem cell differentiation.

35. A method for identifying an agent that modulates stem cell differentiating comprising contacting the agent with a stem cell and a cell culture medium of claim 7 under conditions that favor cell growth and/or differentiation and subsequently contacting the cells with the cell culture medium of claim 1 or 2 under conditions that favor cell growth and/or differentiation, and assaying for stem cell differentiation.
